(19) United States
(12) Patent Application Publication
(10) Pub. No.: US 2021/0215667 A1
(43) Pub. Date: Jul. 15, 2021

(54) MERCURY-FREE P-HYDROXYL PHENYLALANINE DETECTION REAGENT AND PREPARATION METHOD AND APPLICATION THEREOF

(71) Applicant: SHENZHEN HUACHUANG BIO-PHARMACEUTICAL TECHNOLOGY CO., LTD., Shenzhen, Guangdong (CN)

(72) Inventor: Dong LIU, Shenzhen, Guangdong (CN)

(21) Appl. No.: 15/734,139

(22) PCT Filed: Oct. 15, 2018
(86) PCT No.: PCT/CN2018/110242

§ 371 Date: Dec. 1, 2020

(30) Foreign Application Priority Data
Jul. 11, 2018 (CN) 201810757068.2

(51) Int. Cl. G01N 33/493

(52) U.S. Cl. CPC G01N 33/493

(57) ABSTRACT

Disclosed are a mercury-free p-hydroxyl phenylalanine detection reagent and a preparation method and an application thereof. The reagent comprises a buffer solution and tyrosinase and 4-aminoantipyrine which are dispersed in the buffer solution, and may be applied to prepare a kit for p-hydroxyl phenylalanine in urine. The preparation method is to meter and mix the above components. Tyrosinase is used for qualitatively and quantitatively analyzing the p-hydroxyl phenylalanine, and is successfully modified into a producible urine detection reagent. The detection reagent has environment-friendly components, contains no heavy metal ions, such as mercury ions, nickel ions, and cannot cause environment pollutions after use. In addition, the dispersion system is gentle, so that preparation and use processes are quite safe.

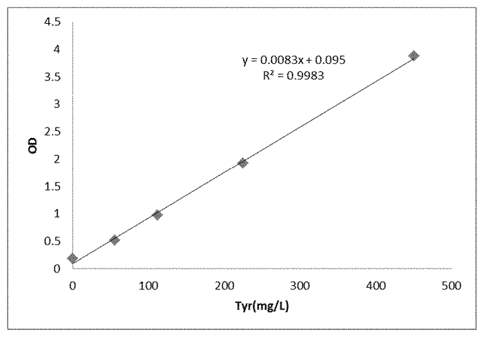

### TECHNICAL FIELD

The present application relates to the field of detection reagents, in particular, to a mercury-free p-hydroxyl phenylalanine detection reagent and preparation method and application thereof.

### BACKGROUND

Early diagnosis and treatment is critical to an increased cure rate of tumors. The current common diagnostic methods including chest X-ray radiography, B-mode ultrasound imaging, computed tomography (CT) and magnetic resonance imaging (MRI) are usually accompanied by procedures such as punctures and blood draws that may aggravate patients' pain or even create a risk of cross-infection. Moreover, these diagnostic methods are expensive, and more importantly, often only able to confirm tumors in the middle and late stages, which offers a significantly reduced cure rate.

Abnormal metabolism of nucleotides in cancer cells produces monohydroxy-phenolic metabolites, among which, p-hydroxyl phenylalanine that can be discharged in urine is present at a particularly higher level, when compared to healthy people. Therefore, measuring the level of p-hydroxyl phenylalanine in a person allows determining whether he/she suffers from a cancer and detecting tumors in early stages. This may save the patient's life without additional expenses while relieving fears and pains from patients.

Existing reagents for testing p-hydroxyl phenylalanine in urine all take advantage of the color development when exposed to mercury (I) or (II) ions. The solution in Chinese Patent application Nos. CN103323452A, CN104535565A, CN106706614A and CN107490689A are all based on this principle. However, this approach is associated with the following disadvantages: (1) the complexation between the amino acid and the metal ions can be significantly interfered with by a high concentration of uric acid or the like in urine, possibly leading to a false negative result; (2) this approach uses mercury ions. The toxicity of mercury ions not only brings a safety risk to preparations of detection reagents but also requires special treatments in producing and recycling processes and thus raises use cost, since metal ions such as mercury ions, and nickel ions contained in liquid waste also do harm to environment; and (3) the use of strong acids such as sulfuric acid and nitric acid also gives rise to many safety issues during the preparation and use processes of such detection reagents.

### SUMMARY

In order to overcome the problem that the existing p-hydroxyl phenylalanine detection reagents all contain mercury and strong acids, the present application provides a mercury-free p-hydroxyl phenylalanine detection reagent and preparation method and application thereof.

To this end, the solution of present application is to provide a mercury-free p-hydroxyl phenylalanine detection reagent comprising a buffer solution and tyrosinase dispersed in the buffer solution.

Optionally, the mercury-free p-hydroxyl phenylalanine detection reagent further comprises 4-aminoantipyrine.

Tyrosinase, also known as polyphenol oxidase, catechol oxidase or the like, can oxidize colorless polyphenols into colored substances such as thearubigens and theaflavins. However, the colors of thearubigens and theaflavins are too light to create a noticeable color gradient, which results in an insufficient sensitivity for the tyrosinase to be used as a urine detection reagent. 4-aminoantipyrine is able to deepen the coloring, thereby enabling the tyrosinase suitable for use as a p-hydroxyl phenylalanine detection reagent.

Optionally, the mercury-free p-hydroxyl phenylalanine detection reagent further comprises sucrose, albumin and Triton X-100 (poly-ethylene glycol octyl phenyl ether), each dispersed in the buffer solution. As a chromogenic reagent, tyrosinase is associated with an unstable storage. Adding appropriate amounts of sucrose, albumin and Triton X-100 to the buffer solution enables to achieve the long-term storage of the detection reagent, which solves the problem in productization of the detection reagent.

Optionally, buffer solution is a phosphate aqueous solution with a pH of 5.0-8.0.

Optionally, tyrosinase is present at a concentration of 50-2000 U/ml.

Optionally, 4-aminoantipyrine is present at a concentration of 0.5-10 mg/ml.

Optionally, sucrose is present at a concentration of 0.02-0.1 g/ml, albumin being present at a concentration of 0.005-0.05 g/ml, Triton X-100 being present at a concentration of 0.005-0.02 g/ml.

In the present application, there is also provided a method for preparing the mercury-free p-hydroxyl phenylalanine detection reagent as defined above. The method comprises steps of: preparing the buffer solution; and dissolving tyrosinase powder and 4-aminoantipyrine in the buffer solution.

In the present application, there is also provided a kit for testing p-hydroxyl phenylalanine in urine. The kit comprises an ampoule containing the mercury-free p-hydroxyl phenylalanine detection reagent as defined above.

Optionally, the mercury-free p-hydroxyl phenylalanine detection reagent contained in the ampoule is present at an amount of 0.1-0.5 ml.

According to the present application, tyrosinase is used to qualitative and quantitative analyze p-hydroxyl phenylalanine and successfully modified into a commercializable urine detection reagent. This detection reagent has environment-friendly components, contains no heavy metal ions, such as mercury ions, nickel ions, and cannot cause environment pollutions after use. In addition, the dispersion system is gentle, so that preparation and use processes are quite safe.

### BRIEF DESCRIPTION OF THE DRAWINGS

The sole FIGURE shows a standard curve in quantitative test experiments by spectrophotometry according to Example 4.

### DETAILED DESCRIPTION

The mercury-free p-hydroxyl phenylalanine detection reagent can be better understood from the following description of several examples. It is to be understood that these examples are presented only to illustrate the present application without limiting the scope thereof.

All the materials used in the following examples were ordinary analytical-grade chemicals, and all the processes were conducted at the room temperature under the atmospheric pressure.

### Example 1

(1) A phosphate buffer solution with a pH of 6.5 was prepared.

(2) 10 KU of tyrosinase dry powder was weighed and dissolved in 10 ml of the phosphate buffer solution having a pH of 6.5, and the preparation of a mercury-free p-hydroxyl phenylalanine detection reagent is completed.

(3) The obtained mercury-free p-hydroxyl phenylalanine detection reagent was dispensed into ampoules at an amount of 0.1 ml per ampoule.

The detection reagent obtained in Example 1 contained the buffer solution and tyrosinase, in which the tyrosinase is present at a concentration of 1000 U/ml.

### Example 2

(1) A phosphate buffer solution with a pH of 7.4 was prepared.

(2) 5 KU of dry tyrosinase powder was weighed and dissolved in 10 ml of the phosphate buffer solution having a pH of 7.4.

(3) 0.1 g of bovine serum albumin, 0.2 g of sucrose and 0.1 g of Triton X-100 were added in the phosphate buffer solution, and the preparation of a mercury-free p-hydroxyl phenylalanine detection reagent is completed.

(4) The obtained mercury-free p-hydroxyl phenylalanine detection reagent was dispensed into ampoules at an amount of 0.1 ml per ampoule.

The detection reagent obtained in Example 2 contained the buffer solution, tyrosinase at a concentration of 500 U/ml, sucrose at a concentration of 0.02 g/ml, albumin at a concentration of 0.01 g/ml and Triton X-100 at a concentration of 0.01 g/ml.

### Example 3

(1) A phosphate buffer solution with a pH of 7.4 was prepared.

(2) 8 KU of tyrosinase dry powder was weighed and dissolved in 10 ml of the phosphate buffer solution having a pH of 7.4.

(3) 0.01 g of 4-aminoantipyrine was added in the phosphate buffer solution.

(4) 0.1 g of bovine serum albumin, 0.2 g of sucrose and 0.1 g of Triton X-100 were added in the phosphate buffer solution, and the preparation of a mercury-free p-hydroxyl phenylalanine detection reagent is completed.

(5) The obtained mercury-free p-hydroxyl phenylalanine detection reagent was dispensed into ampoules at an amount of 0.1 ml per ampoule.

The detection reagent obtained in Example 3 contained the buffer solution, tyrosinase at a concentration of 800 U/ml, 4-aminoantipyrine at 1 mg/ml, sucrose at 0.02 g/ml, albumin at 0.01 g/ml and Triton X-100 at 0.01 g/ml.

### Example 4: Experiments for Performance Demonstrations

4.1 Qualitative Testing Experiments

Aqueous solutions of p-hydroxyl phenylalanine at increasing concentrations respectively of 0 mg/L, 60 mg/L, 120 mg/L, 250 mg/L and 450 mg/L were added to the detection reagents prepared in the above Examples, and the resulting color changes were recorded and summarized in Table 1.

TABLE 1

| | | Example 1 | Example 2 | Example 3 |
|---|---|---|---|---|
| Initial Color of | | Colorless | Colorless | Light Yellow |
| Detection Reagent | | | | |
| Color | 0 mg/L | Colorless | Colorless | Light Yellow |
| change | 60 mg/L | Light Yellow | Light Yellow | Light Red |
| after | 120 mg/L | Orange Yellow | Orange Yellow | Red |
| addition | 250 mg/L | Dark Orange | Dark Orange | Dark Red |
| of testing | | Yellow | Yellow | |
| sample | 450 mg/L | Dark Orange | Dark Orange | Dark Red |
| | | Yellow | Yellow | |

As can be found from Table 1, after testing samples containing the p-hydroxyl phenylalanine are added, the detection reagents according to the present application experienced a remarkable color change, and the higher the concentration of p-hydroxyl phenylalanine was, the more the color changed.

4.2 Quantitative Testing Experiments by Spectrophotometry

In addition to the quantitative analysis by naked-eye observation, solutions of the detection reagent obtained in Example 3 were also analyzed by spectrophotometry after reaction as 4-aminoantipyrine was added, and the results are shown in the sole FIGURE. A standard curve may be plotted from tyrosine solutions with varying concentrations. A quantitative concentration of a p-hydroxyl phenylalanine testing sample can be obtained from a measured absorbance value of the testing sample according to the standard curve.

4.3 Experiments on Stability

The reagents of Examples 1-3 were placed in an oven at 50° C. for one week, and then tested respectively. The results show that, at a low concentration of p-hydroxyl phenylalanine, the reagent of Example 1 did not exhibit a color change, while reagents of Example 2 and 3 maintain color change performances.

4.4 Experiments on Anti-Interference Performance

A 250 mg/L p-hydroxyl phenylalanine aqueous solution was prepared, in which uric acid is contained at 600 μmol/L, and tested with the reagents of Examples 1, 2 and 3 according to the present application, as well as with a reagent prepared according to Chinese Patent application. No. CN104535565A. As a result, each of the reagents of Examples 1, 2 and 3 exhibited a color change, while the reagent according to CN104535565A did not exhibit a color change.

At last, it is to be noted that the foregoing examples are presented merely to illustrate the technical solution of the present application and do not limit it in any sense. Although the present application has been described in detail with reference to the above examples, it is to be understood that modifications or equivalent substitutions of all or some of the features to the technical solution of foregoing examples can be made by those of ordinary skill in the art, and such modifications or substitutions do not make essences of the corresponding technical solutions departing from the scope of the technical solutions of the foregoing examples of the present application.

1. A mercury-free p-hydroxyl phenylalanine detection reagent comprising a buffer solution and tyrosinase dispersed in the buffer solution.

2. The mercury-free p-hydroxyl phenylalanine detection reagent according to claim 1, wherein the buffer solution further has 4-aminoantipyrine dispersed therein.

3. The mercury-free p-hydroxyl phenylalanine detection reagent according to claim 2, wherein 4-aminoantipyrine is present at a concentration of 0.5-10 mg/ml.

4. The mercury-free p-hydroxyl phenylalanine detection reagent according to claim 1, wherein the buffer solution is a phosphate aqueous solution with a pH of 5.0-8.0.

5. The mercury-free p-hydroxyl phenylalanine detection reagent according to claim 1, wherein tyrosinase is present at a concentration of 50-2000 U/ml.

6. The mercury-free p-hydroxyl phenylalanine detection reagent according to claim 1, further comprising sucrose, albumin and Triton X-100, each of which is dispersed in the buffer solution.

7. The mercury-free p-hydroxyl phenylalanine detection reagent according to claim 6, wherein sucrose is present at a concentration of 0.02-0.1 g/ml, and wherein albumin is present at a concentration of 0.005-0.05 g/ml and Triton X-100 is present at a concentration of 0.005-0.02 g/ml.

8. A method for preparing the mercury-free p-hydroxyl phenylalanine detection reagent of claim 1, comprising steps of: preparing the buffer solution; and dissolving other component(s) in the buffer solution.

9. A kit for testing p-hydroxyl phenylalanine in urine, comprising an ampoule containing the mercury-free p-hydroxyl phenylalanine detection reagent of claim 1.

10. The kit according to claim 9, wherein the mercury-free p-hydroxyl phenylalanine detection reagent contained in the ampoule is present at an amount of 0.1-0.5 ml.
